(19) United States
(12) Patent Application Publication
(10) Pub. No.: US 2013/0261540 A1
(43) Pub. Date: Oct. 3, 2013

(54) HIGH-PRESSURE PNEUMATIC INJECTION SYSTEM AND METHOD

(71) Applicant: Justin M. Crank, Maple Grove, MN (US)

(72) Inventor: Justin M. Crank, Maple Grove, MN (US)

(21) Appl. No.: 13/994,285

(22) PCT Filed: Dec. 16, 2011
(86) PCT No.: PCT/US11/65510

§ 371 Date: Jun. 14, 2013

Related U.S. Application Data
(60) Provisional application No. 61/423,873, filed on Dec. 16, 2010.

(51) Int. Cl. A61M 5/30

(52) U.S. Cl. CPC A61M 5/30
USPC 604/70

(57) ABSTRACT

A needleless or high-pressure injection system for injecting treatment or therapeutic fluid to tissue of the lower urinary tract, such as the prostate or bladder, is provided. The systems or devices can release the therapeutic fluid or “injectate” at high-pressure out of one or more orifices at the end of an elongate shaft inserted into the urethra. Various embodiments can include a pneumatic or fully-pneumatic system adapted to selectively release the high-pressure injectate, while also providing firing or triggering protection.

### PRIORITY

This application claims priority to and the benefit of U.S. Provisional Patent Application No. 61/423,873, filed Dec. 16, 2010, which is incorporated herein by reference in its entirety.

### FIELD OF THE INVENTION

The invention relates generally to surgical tools and methods and, more particularly, to methods and devices for treating tissue of the urinary tract (e.g., prostate tissue, kidneys, ureters, urethral tissue, bladder, etc.), using needleless or high-pressure jet injection devices for injecting fluid into tissue.

### BACKGROUND OF THE INVENTION

Lower urinary tract health is an increasingly important health issue, e.g., based on an aging population. Treatment of lower urinary tract conditions is an area of much investigation. Prostate disease, for example, is a significant health risk for males. Diseases of the prostate include prostatitis, benign prostatic hyperplasia (BPH, also known as benign prostatic hypertrophy), and prostatic carcinoma.

Prostatitis is an inflammation of the prostate gland. Types include acute and chronic bacterial forms of prostatitis, and a non-bacterial form. Symptoms can include difficult urination, burning or painful urination, perineal or lower back pain, joint or muscle pain, tender or swollen prostate, blood in the urine, or painful ejaculation. Prostatitis is caused by bacterial infection in many instances, in which case treatment generally includes antimicrobial medication. Noninfectious forms of prostatitis are treated by other means such as administration of an alpha-1-adrenoreceptor antagonist drug to relax the muscle tissue in the prostate and reduce the difficulty in urination.

Benign prostatic hypertrophy (BPH) is a very common disorder affecting an estimated 12 million men in the United States alone. BPH is a chronic condition and is strongly age-related; approximately 50% of men over the age of fifty, 75% of men beyond the age of seventy, and 90% of men over the age of eighty are afflicted with BPH. BPH is a non-cancerous condition characterized by enlargement of the prostate, obstruction of the urethra, and gradual loss of bladder function. Symptoms include difficult urination, frequent urination, incomplete emptying of the bladder, and urgency.

BPH may be treated with a number of therapeutic modalities including surgical and medical methods, depending on severity of symptoms. Treatments range from “watchful waiting” for men with mild symptoms, to medications, to surgical procedures. Examples of useful medications include 5-alpha reductase inhibitors such as Avodart™ and Proscar®.

Transurethral resection of the prostate (TURP) is a preferred surgical method of treating BPH. A typical TURP procedure requires general anesthesia and the placement of a resectoscope in the urethra for removal of multiple small chips of hyperplastic prostatic tissue to relieve the obstruction. Complications from TURP include bleeding, incontinence, retrograde ejaculation, and impotence.

An alternate surgical method for treating BPH is transurethral incision of the prostate (TUIP). In the TUIP procedure, incisions are made in the prostate to relieve pressure and improve flow rate. Incisions are made where the prostate meets the bladder. No tissue is removed in the TUIP procedure. Cutting muscle in this area relaxes the opening to the bladder, which decreases resistance to urine flow from the bladder. A variant of the TUIP procedure in which a laser is used to make the incision is known as transurethral laser incision of the prostate (TULIP).

Other surgical methods used to relieve the symptoms of BPH include methods of promoting necrosis of tissue that blocks the urethra. Hyperthermic methods, for example, use the application of heat to “cook” tissue and kill the cells. The necrosed tissue is gradually absorbed by the body. Several methods of applying heat or causing necrosis have been demonstrated, including direct heat (transurethral needle ablation, or TUNA), microwave (transurethral microwave treatment, or TUMT), ultrasound (high-intensity focused ultrasound, or HIFU), electrical vaporization (transurethral electrical vaporization of the prostate, or TUEVP) and laser ablation (visual laser ablation of the prostate, or VLAP), among others.

Chemical ablation (chemoablation) techniques for promoting prostate tissue necrosis have also been considered. In one chemical ablation technique, absolute ethanol is injected transurethrally into the prostate tissue. This technique is known as transurethral ethanol ablation of the prostate (TEAP). The injected ethanol causes cells of the prostate to burst, killing the cells. The prostate shrinks as the necrosed cells are absorbed.

In addition to prostate conditions, other tissue of the urinary tract can be affected by medical conditions that can be treated by delivery of various therapeutic materials in the form of fluids. Tissues of the bladder (which includes the bladder neck), ureter, kidneys, urethra, as well as the prostate, can be treated by delivery of drugs or other therapeutic agents, such as botox.

Therapeutic agents should be delivered with minimized discomfort and procedure time, and with the best degree of accuracy of delivery location and delivery volume as possible. As such, there exists continuing need to provide improved devices for delivering therapeutic fluids to the lower urinary tract, kidneys, ureters, etc.

### SUMMARY OF THE INVENTION

The invention relates generally to needleless or high-pressure injection devices useful for injecting fluid to tissue of the lower urinary tract, such as the prostate or bladder. The devices inject a therapeutic fluid or “injectate” at high-pressure using one or more orifices at the end of an elongate shaft inserted into the urethra. To treat the prostate, the injectate fluid can pass through the urethra and disperses in the prostate as a cloud of particles. In addition, devices of the present description can be useful to treat tissue of the urinary tract in females or males. For example, devices of the invention may be useful to inject the bladder, bladder neck, the urethral tissue itself or the external sphincter, or for transurethral injection of the prostate in a male. In other embodiments, a fluid may be injected into tissue of the urinary tract (e.g., bladder, urethra, kidneys, ureters, prostate, etc.) such as individual or combination treatments using drugs or other therapeutic agents, e.g., botulinum toxin, an antiandrogen, among others as will be understood.

The needleless systems can overcome undesired or disadvantageous features of systems and methods that use a needle, e.g., for transurethral injections of fluid into the prostate or the bladder. A needleless mode of injecting a fluid into the prostate or other tissue of the lower urinary tract requires that certain technical challenges be overcome to accommodate the specific technical and medical needs of injecting a therapeutic fluid to internal tissue, optionally transurethrally, without a needle. For instance, to inject the prostate, a needleless injector must be of a size and shape that may be placed within the urethra while also providing an injectate at the injection orifice in the prostatic urethra at a pressure sufficient to penetrate urethral and prostate tissues. The injectate must penetrate urethral and prostate tissues in a predictable and desired fashion to become dispersed throughout the tissue.

Features of needleless injector devices described above are included as part of the present disclosure and may be included in a needleless injector device individually or in any desired combination. For example, embodiments of the invention can include needleless injector devices that include positioning features that facilitate proper positioning of an injection orifice in the urethra. Positioning features are various in nature and may include one or more of: a balloon or multiple balloons located at the distal end of the device for placement and fixing the distal end; multiple orifices; moveable orifices; demarcation of distances to distal end features, at the proximal end; and an optical feature such as an endoscope or optical fiber. Other embodiments of needleless injector devices include the above features along with one or more tissue tensioners that contact and optionally place pressure on tissue at a desired location relative to an injection orifice, and optionally can also place a strain or tension on the tissue as desired for delivery of an injection at the surface of the tissue. Examples of tissue tensioners include inflatable or extendable features such as balloons or mechanically extendable features such as paddles, metal cages, other mechanically extendable protrusions, a vacuum, etc.

Needleless injector devices as described can be used with various delivery methods such as methods that allow for direct vision of an injection wherein an internal location of an injection orifice is determined visually, and methods referred to as blind delivery methods wherein location of an injection orifice is determined indirectly. Direct vision methods can involve the use of an optical feature to view an injection site directly, such as by use of an endoscope or optical fiber that is included in an injector device, e.g., as a component of the shaft. A device that allows for blind delivery can instead include one or more non-optical features that allow a surgeon to identify the position of a device, and in particular an injection orifice, e.g., within the urethra, so that an injection can be performed at a desired location. Blind delivery techniques can identify a delivery location based on features of the device such as a length-measuring feature such as demarcations at the proximal end of the device that reference locations and provide visualization of features at the distal end, by using demarcations in combination with known dimensions of a device and of anatomy. Demarcations may be used also in combination with measurement of anatomical features such as the length of the prostate, e.g., by known techniques including those that use ultrasound or x-ray position measuring equipment. Blind delivery techniques can also involve other features of devices as described herein such as positioning features (e.g., balloons at the distal end of the device) and moveable injection orifices.

Embodiments can include a pneumatic system to control pressure and firing of the needleless device. The pneumatic system can include an automated balloon control system and an automated injection control system. Each of the control systems can include a lock-out circuit or feature. The lock-out circuit of the automated balloon control system is adapted to control inflation and deflation of a balloon element. The lock-out circuit of the automated injection control system is adapted to control high-pressure release of a fluid treatment injectate from one or more injectate orifices at an end of the needleless device.

### BRIEF DESCRIPTION OF THE DRAWINGS

FIG. 1 illustrates a needleless injection catheter system in accordance with embodiments of the present invention.

FIGS. 2-4 illustrate distal ends of a needless injection catheter system having injectate orifices and a balloon element in accordance with embodiments of the present invention.

FIG. 5a is a stage or state diagram for a balloon control system in a pneumatic system in accordance with embodiments of the present invention.

FIG. 5b is a stage or state diagram for an injection control system in a pneumatic system in accordance with embodiments of the present invention.

FIGS. 6-7 are block diagrams of injection control systems and circuitry for pneumatic systems in accordance with embodiments of the present invention.

FIG. 8 is a block diagram of an injection control system and circuitry for a pneumatic system in accordance with embodiments of the present invention.

### DETAILED DESCRIPTION OF PREFERRED EMBODIMENTS

The invention relates to devices and methods useful for injecting fluid into tissue for treatment. The fluid can be injected without the use of a needle and can therefore be referred to as a needleless fluid injection system. Needleless fluid injection systems of the invention can include one or more orifices that deliver fluid in the form of a stream of fluid, which may be referred to as a jet or fluid stream, at a pressure, velocity, and stream size that allow the fluid stream to pass through a tissue surface, penetrate into the bulk of the tissue below the tissue surface, and become dispersed as fluid particles within the tissue, such as in the form of a cloud of dispersed fluid particles or droplets, without a needle structure passing into the tissue. The type of tissue injected for treatment can be any amenable tissue, which can include tissue at or near the urinary tract (e.g., tissue of the prostate, kidneys, ureters, urethral tissue, bladder, bladder neck, etc.), or other tissues such as heart tissue, as desired. The fluid delivered can include drugs or other therapeutic agents (chemical or biologic).

Needleless devices of the type described herein generally include a distal end and a proximal end. As used herein, a “distal end” of a device or system refers to an end area or portion of the device or system that can be introduced internally within a patient's body during a treatment procedure, generally including the distal end of an elongate shaft or catheter tube. For example, an elongate shaft or catheter of the needleless injection systems of the invention generally includes a distal end that is the first portion of the device that is introduced into the patient for treatment. A distal end may include functional features that operate on fluid or tissue during use, such as one more ejection orifices and delivery heads (e.g., end effectors, nozzles, etc.), a frictional tissue holding tip, placement devices, tissue tensioners, lighting or other optical features, steering features, and the like.

As used herein, a “proximal end” of an exemplary needleless device or system is the end that is opposite the distal end of that device or system. Each individual component of a system can include its own proximal and distal ends, while the overall system can also include proximal and distal ends. For one example, a needleless fluid injection system of the invention can include an injector body or console at a proximal end that remains external to the patient during use and an elongate shaft or catheter tube at a distal end. That is, exemplary needleless fluid delivery devices or systems can include a proximal end that includes a console, and an elongate shaft extending from a proximal end, which is in communication with the console, to a distal end. One or more injection orifices at the distal end can be in fluid communication with the console.

Various device structures, components, mechanisms, methods and techniques described and depicted in U.S. Patent Publication No. 2006/0129125 and International Publication Nos. WO2011/011423, WO2011/011382, WO2010074705 and WO2007/079152 are envisioned for use, alone or in combination, with the present invention. As such, the entire disclosures of the above-referenced publications are incorporated herein by reference.

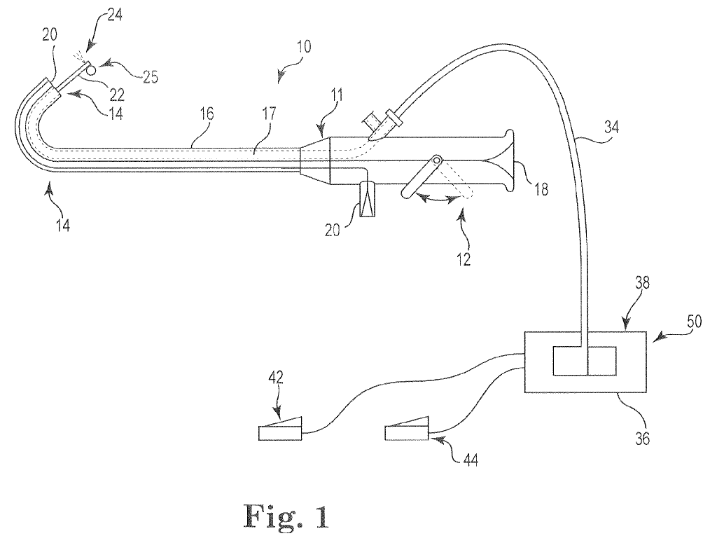

In one embodiment, such as that shown in FIG. 1, an injection catheter system 10 can include a needless injection device 11 having a proximal portion 12 and a distal portion 14, and a shaft or body portion 16 extending there between. The proximal portion 12 generally includes a handle 18, and a connection port or assembly adapted to interconnect with a fluid source 36. The fluid source 36 is in operative and fluid communication with the proximal portion 12 via a conduit 34. The fluid source 36 can include a reservoir for a therapeutic drug, agent or like substances or therapeutic fluids making up the treatment injectate, and a pressure source capable of pressurizing and advancing fluid contained in the source, as well as the pneumatic components and circuitry adapted to control the pressure and ejection of the injectate. The fluid source 36 can be generally “remote” (FIG. 1) from the proximal portion or the distal portion 14, or provided generally proximate or directly attached to the device components.

The therapeutic fluids can include biologically active species and agents such as chemical and biochemical agents, for example. Exemplary devices are designed to deliver fluid at various tissue locations, and can further deliver multiple different therapeutic fluids having varying material properties (e.g., viscosity) using a single system. The devices can be capable of delivering precise amounts of fluid for injection at precise locations and at specific pressures to a location in the patient.

In certain embodiments where the fluid source 36 is remote or separated from, but in operable communication with, the injection device 11, the fluid source 36 can be housed or otherwise provided in or with a console 38. An exemplary console 38 used with systems of the invention can include a housing that connects to or is otherwise (directly or indirectly) in fluid and operable communication with the device 11. The console 38 can include fluid that can be pressurized by a pressure source, such as CO_{2,} to cause the fluid to flow through the shaft for injection into tissue at the distal end 14. The device 11 can eject fluid from one or more ejection orifices 24 that can be located at the distal end 14 of the shaft or catheter tube. Optionally, multiple injection orifices may be located at one or more locations along a length of or about a circumference of a shaft distal end. Devices, systems, and methods are described herein that can be used to inject a fluid through a surface of a tissue, penetrating without the use of a needle through the tissue surface and into the bulk of the tissue, and dispersing as particles or droplets within the tissue below the tissue surface. The injected fluids may be referred to as an “injectate” or “injection fluid,” which may be any type of fluid such as a therapeutic fluid. The injectate can be administered into tissue in a needleless manner, whereby the injectate is delivered as a pressurized fluid stream or jet. This contrasts with injections performed using a needle, whereby a hollow needle structure penetrates tissue to locate a hollow end of the needle within a tissue mass, below the tissue surface, after which the needle carries fluid into the bulk of the tissue and delivers the fluid at a relatively low pressure to the tissue in the form of a body or pool of fluid known as a bolus.

A working lumen or channel 17 extends within the shaft 16 and contains a fluid delivery lumen 22 such that the lumen 22 is adapted to move longitudinally along the length of the body 16 to allow the distal end of the fluid delivery lumen 22 to extend from the tip of the distal portion 14 as an orifice extension. The high-pressure injectate is delivered to the target tissue from the fluid delivery lumen 22. In particular, the injectate traverses from the fluid source 36, into the working channel 17, and out of the fluid delivery lumen 22, due to the pressure provided by the console 38. The shaft 16 can include a fiber optic feature 20, e.g., an endoscope device, having a light source to transmit light to the distal portion 14.

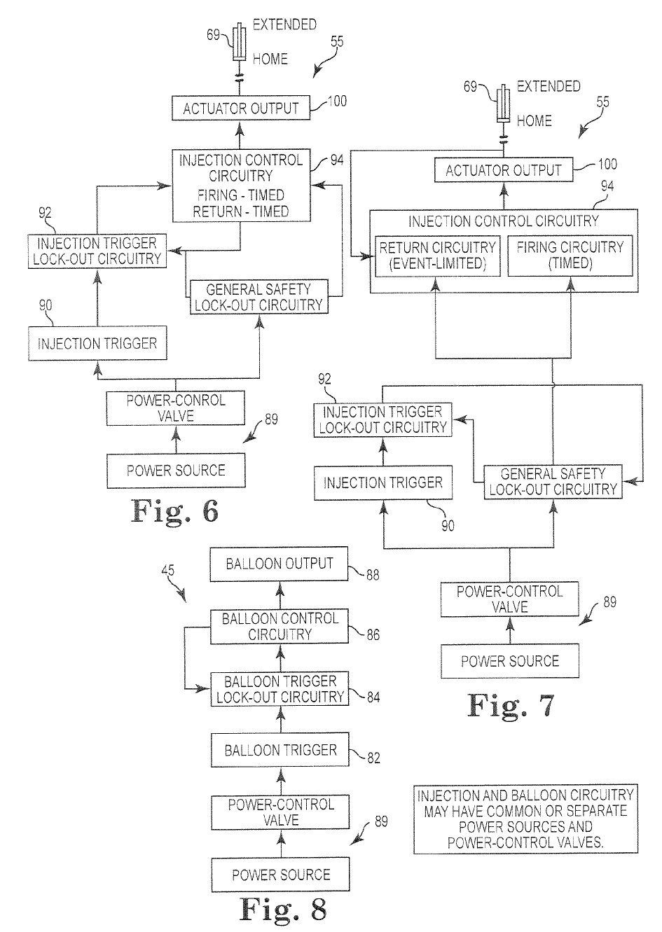

In one embodiment, the fluid source 36 and console 38 can include a fully-pneumatic pressure system. Power for the pneumatic system can come from a CO₂tank or source (see FIGS. 6-8). The pressure of the system can be monitored by a gauge.

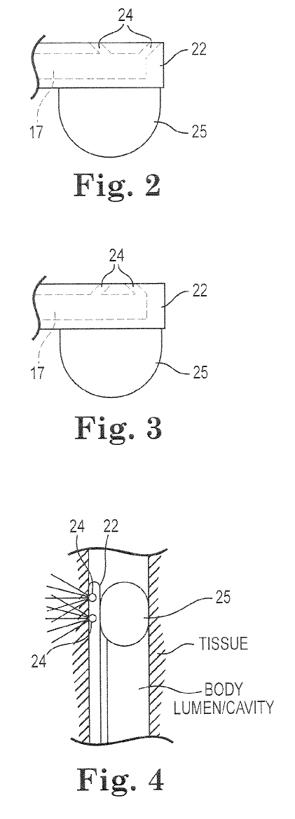

Additional examples (in side cross-section) of distal ends of an injection shaft 22 are shown in FIGS. 2-4 that involve an injection force that is opposed by a control force in use. The control force can be produced by inflation of a balloon (or other apposition device or mechanism) 25. A resultant generally opposing control force (to the ejection force) is preferably provided by the balloon 25. Other apposition devices can be used in addition to or in place of balloon 25, including one or more control orifices. Various embodiments can exclude an opposition device or force all together. As shown, injection orifices 24 are longitudinally spaced apart along the length of the injection shaft 22 and may be positioned relative to the injection shaft in any desired manner, including being spaced around the circumference of the injection shaft 22 and at any desired angle relative to the longitudinal axis of the shaft.

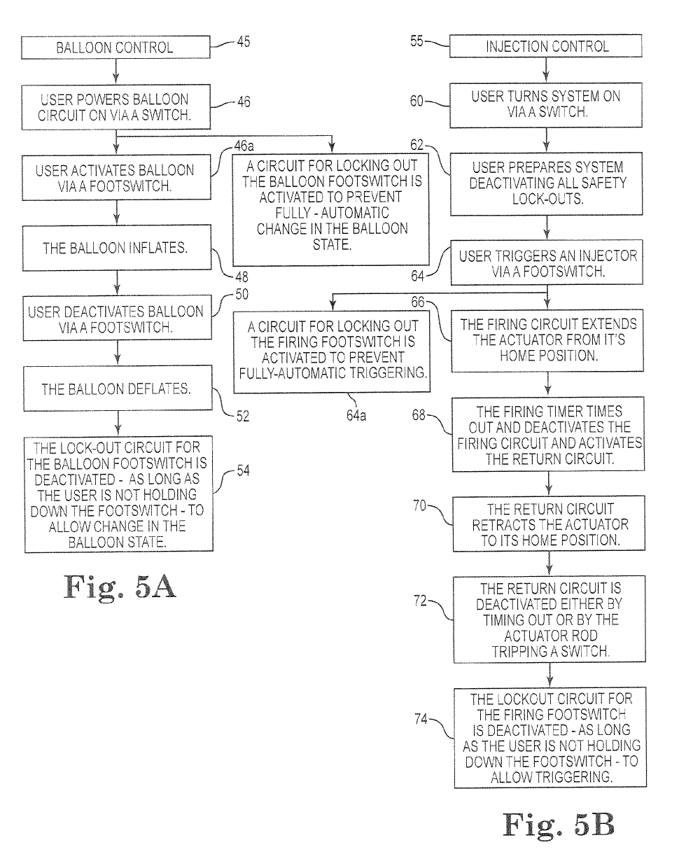

The pneumatic system can include various components, regulators, valves and pneumatic circuitry elements adapted to facilitate pressure cycles to trigger or fire the injectate out through the one or more orifices 24 of the device 11. In certain embodiments, such as those shown in FIGS. 5a-8, the system can include one or more actuating mechanisms 42 to provide user control for triggering the injectate fire cycle, and one or more actuating mechanisms 44 to control the inflation and deflation of the balloon element 25. As shown in FIG. 1, the injectate actuator 42 and the balloon actuator 44 can include a foot pedal or like switch device. Other embodiments can use hand triggers, buttons, switches or various other electrical, mechanical or pneumatic devices or mechanisms known to one of ordinary skill in the art.

In addition, as shown in FIGS. 5a-8, the pneumatic system can include a balloon control system 45 and an injection control system 55 to provide automated control, safety and use of certain portions of the pneumatic system. The balloon control system is initiated or triggered via the operably connected foot pedal actuator 44 and the injection control system 55 is initiated or triggered by the operably connected foot pedal actuator 42.

Exemplary operational stages or steps of the balloon control system 45 are depicted in FIG. 5a. Exemplary operational stages or steps of the injection control system 55 are depicted in FIG. 5b. Further, block diagrams of the pneumatic circuits for embodiments of the invention are provided in FIGS. 6-8.

For the balloon control system 45, the operation starts at a user power stage 46, where the user generally initiates a power source 89, as shown in FIGS. 5a and 8. The user then actuates the foot switch or pedal 44 at stage 46a to initiate a balloon trigger circuit 82 which, in turn, initiates the balloon 25 inflation stage 48 and balloon control circuitry 86 by allowing gas to flow through to the balloon 25. Further, actuation of the switch 44 activates the balloon lock-out circuit 84 within the system 45 to prevent undesirable, automatic, changes in the balloon construct via the pedal 44 after inflation is achieved. The balloon output 88 is in operable communication with the balloon 25. Releasing the pedal 44 still retains pressure in the balloon 25 to keep it inflated. When the user later wants to deflate the balloon 25 to reposition the distal end 14 and the corresponding injectate orifices 24, or to remove the device 11 from the patient upon completion of the treatment procedure, the user simply re-engages or activates the foot pedal 44 at deactivation stage 50 to initiate deflation of the balloon at stage 52. At this point, as long as the pedal 44 is not being held down, the lock-out circuit 84 for the balloon circuit deactivates. Further embodiments of the balloon lock-out circuit 84 are depicted in the block diagram of FIG. 8. Obviously, variations, modifications or substitutes to the specifically depicted and described circuit structures and components are envisioned as well to achieve the steps, operations and protections taught herein for the balloon control system 45, and the injection control system 55.

Exemplary operational stages or steps, of the injection control system 55 are depicted in FIG. 5b. FIGS. 6-7 provide block diagrams of embodiments of the injection control system 55 circuitry. The trigger event is actuation of the pedal 42 to control extension of an actuator or member 69, driven by power source 89, to facilitate the release of high pressure injectate from the orifices 25. After turning the system on at power stage 60 to initiate the power source 89, the user can prepare the system for use at stage 62. Assuming the balloon 25 is properly placed and inflated to provide the proper counter force to maintain the injectate orifices 24 at the proper target treatment site (in those embodiments have a balloon 25), the user can proceed to stage 64 by activating the switch or foot pedal 42 to initiate the injection trigger 90 and trigger the injection control circuitry 94 to eventually activate or extend the actuator 69 to drive the injectate from the orifices 24. Initiating the pedal 42, in turn, also activates the system 55 circuit lock-out circuitry 92 at stage 64a, to prevent certain undesirable injectate firing or triggering responses. For instance, embodiments can include a footswitch lock-out circuit 92 (e.g., shown in FIGS. 6-7) to prevent hang firing or repetitive firing of injectate out through the orifices 25 even if the pedal 42 is continuously held down or engaged for a prolonged period of time. Embodiments of the lock-out circuit 92 can prevent triggering if the system is already in a firing stroke (extending actuator 69), a return stroke (actuator 69 returning to home position) or if the foot pedal 42 is being continuously held down or engaged. Various different lock-out circuits 92 are envisioned to provide these protections. For instance, the circuit 92 of FIG. 7 is event driven such that the position of the actuator 69 (e.g., extended position) is known. As such, input from the pedal 42 is disabled to prevent additional firing until the actuator 69 returns to its home position, or just before reaching its home position. Alternatively, as shown in FIG. 6, the lock-out circuit 92 can be time driven such that a predefined time (e.g., seconds) is built into the system in which input from the foot pedal 42 is disabled or disallowed. In such embodiments, the cycle can be in the range of 3 to 10 seconds, approximately 5 seconds, or a myriad of other cycle or firing durations depending on the particular treatment parameters and procedure goals.

Assuming a proper actuation of the pedal 42, at stage 66, the injectate firing circuit extends the actuator 69 out from its home or initial rest position to facilitate ejection of the injectate from the orifices 25 under high pressure (e.g., firing stroke). At a predefined time (seconds), the firing times out or deactivates at stage 68, thus reducing pressure and returning the actuator 69 to its home or rest position at stage 70 (e.g., return stoke). Full or nearly full return of the actuator 69 to the home position trips a switch, or otherwise provides a time-out event, at stage 72 to indicate a return stroke. As such, the lock-out circuit 92 is deactivated at stage 74 as long as the user is not at that time holding down the foot switch or pedal 42. Then, the system 55 can again be triggered to release a new round of injectate as desired.

The lock-out circuits work by receiving an initial input from the foot pedal or switch, then diverting subsequent inputs (injection or balloon) inputs in a way that prevents the change of state necessary for the foot pedal or switch to function again. As a result, holding down the foot pedals or switches will not cause rapid fire (machine-gun-like) injection releases or rapid cycling between inflation and deflation of the balloon.

A broad stop switch, pedal, button or like mechanism or device can be included to provide an overriding safety feature to kill the entire system. Further, injection control system 55 and the balloon control system 45 can share a common power source 89, such as CO₂, or each system 45, 55 can have its own separate power source.

Various embodiments, such as that depicted and described for FIG. 3, can include a fully-pneumatic means to activate an injection or injectate catheter with a system cycle having an expelling and resetting stroke. The activation can occur with actuation of a balloon or the footswitch. The injection console can be activated and controlled without any electronic components. The injection console can automatically control the actuation of the injector as well as the inflation/deflation of the balloon. The power and dose can be manually controlled by regulator and dose-selectors. A single footswitch can be pressed once so that the actuator cycles through its firing and returning strokes. Before firing, the return side of the actuator can be vented. The desired control of the balloon is to press a single footswitch once to inflate the balloon and a second time to deflate the balloon. As such, electrical power and software is not required.

In certain embodiments, as depicted and described, the system 10 can include both an automated balloon inflation system 45 and an automated injection control system 55. The system 10 can include an automated injection control system 55 without an automated balloon inflation system 45 (e.g., manual balloon inflation). Further, there are embodiments where the system 10 will not include a balloon 24 at all, but will still include an automated injection control system 55. Various other iterations and combinations of these systems and features are envisioned for use with embodiments of the present invention.

A variety of materials may be used to form portions or components of the system 10, including nitinol, polymers, elastomers, fluid systems and components, thermoplastic elastomers, metals, ceramics, circuitry, springs, wires, tubing, and the like. The system 10, and its components, devices, systems and methods may have a number of suitable configurations known to one of ordinary skill in the art.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is, therefore, to be understood that within the scope of the appended claims, the invention may be practiced other than as specifically described herein.

1. A high-pressure needleless injection system, comprising:
an injection device including a proximal end portion and a distal end portion, the distal end portion including one or more injectate orifices and an inflatable balloon element;
a pneumatic pressure system including;
a balloon control system adapted to selectively control inflation and deflation of the balloon element, the balloon control system having a lock-out circuit to prevent rapid cycling between an inflated and deflated state for the balloon element; and
an injection control system adapted to selectively control high-pressure release of a fluid treatment injectate from the one or more injectate orifices, the injection control system having a lock-out circuit to prevent a rapid fire state of the fluid treatment injectate from the one or more injectate orifices.

2. The system of claim 1, wherein the balloon control system further includes a balloon activation switch.

3. The system of claim 2, wherein the balloon activation switch is a foot pedal actuator switch.

4. The system of claim 1, wherein the injection control system further includes all injection activation switch.

5. The system of claim 4, wherein the injection activation switch is a foot pedal actuator switch.

6. The system of claim 1, wherein at least one of the balloon control system and the injection control system are provided in a console remote from and in operable communication with the injection device.

7. The system of claim 1, wherein at least one of the balloon control system and the injection control system are provided directly with and in operable communication with the injection device.

8. The system of claim 1, wherein the balloon control system and the injection control system are provided in a console remote from and in operable communication with the injection device.

9. The system of claim 1, wherein the fluid treatment injectate includes a drug.

10. The system of claim 1, wherein the fluid treatment injectate includes a treatment agent.

11. The system of claim 1, wherein the lock-out circuit of the injection control system prevents input from an activation switch if a return firing state is not achieved.

12. The system of claim 1, wherein the fluid treatment injectate is adapted to treat tissue of the prostate.

13. A high-pressure needleless injection system, comprising:
an injection device including a proximal en portion and a distal end portion, the distal end portion including a plurality of injectate orifices, and an opposing inflatable balloon element; and
a pneumatic pressure system including an input switch and an injection control system adapted to selectively control high-pressure release of a fluid treatment injectate from the one or more injectate orifices, the injection control system further having a lock-out circuit to prevent a rapid fire state of the fluid treatment injectate front the plurality of injectate orifices by preventing input from the input switch when the injection control system is in a firing state.

14. The system of claim 13, wherein the pneumatic pressure system further includes a balloon control system adapted to selectively control inflation and deflation of the balloon element.

15. The system of claim 14, wherein the balloon control system further includes a lock-out circuit to prevent rapid cycling between an inflated and deflated state for the balloon element.

16. The system of claim 13, wherein the input switch is a foot pedal actuator switch.

17. The system of claim 13, wherein the injection control system is provided in a console remote from and in operable communication with the injection device.

18. The system of claim 13, wherein the fluid treatment injectate includes a drug.

19. The system of claim 13, wherein the fluid treatment injectate includes a treatment agent.

20. The system of claim 13, wherein the fluid treatment injectate is adapted to treat tissue of the prostate.
